(19) United States
(12) Patent Application Publication
(10) Pub. No.: US 2023/0048236 A1
(43) Pub. Date: Feb. 16, 2023

(54) Health Inventory

(71) Applicant: Jedlyn Pierrilus, West Palm Beach, FL (US)

(72) Inventor: Jedlyn Pierrilus, West Palm Beach, FL (US)

(21) Appl. No.: 17/402,577

(22) Filed: Aug. 15, 2021

(51) Int. Cl. G16H 10/60; G16H 80/00

(52) U.S. Cl. CPC G16H 10/60; G16H 80/00

(57) ABSTRACT

A process to securely store and manage patient healthcare information. The patient provides some information that is available to selected healthcare providers thereby avoiding repeating the same information to each treating healthcare provider. Healthcare providers may also store information about the patient on the system to make that information available to other treating healthcare providers. The information is owned by the patient and is portable with the patient.

### B. CROSS-REFERENCES TO RELATED APPLICATIONS

None.

### C. STATEMENT REGARDING FEDERAL SPONSORED RESEARCH OR DEVELOPMENT

None.

### D. NAMES OF PARTIES TO A JOINT RESEARCH AGREEMENT

None.

### E. REFERENCE TO A “SEQUENCE LISTING”, A TABLE, OR A COMPUTER PROGRAM LISTING APPENDIX SUBMITTED ON COMPACT DISC AND INCORPORATION-BY-REFERENCE OF THE MATERIAL ON THE COMPACT DISCLOSURE

None.

### F. STATEMENT REGARDING PRIOR DISCLOSURES BY AN INVENTOR OR JOINT INVENTOR

None.

### G. BACKGROUND OF THE INVENTION

### 1. Field of the Invention

The present invention relates to improving healthcare, and more particularly, to an improved device and method of use to maintain health information and improve the provision of healthcare.

### 2. Description of Related Art Including Information Disclosed Under 37 Cfr 1.97 and 37 Cfr 1.98

Several designs for health information management have been designed in the past. None of them, however, includes a consolidated technological solution and method of managing patient information where a team of healthcare providers and the patient herself can collaborate with and access up to date and comprehensive information.

Some hospitals and hospital systems may maintain patient charts that have information from multiple medical disciplines. However, these repositories of medical information are not portable with the patient. When the patient seeks an outside service provider, that provider may not have access to the hospital system records.

For example, a patient may move from one region to another, or be traveling, and therefore records from one regional hospital or urgent care system may not be available to another regional system. A system is needed where patient records are owned by the patient and remain portable with the patient regardless of where on earth the patient is seeking health care services.

Other patents and devices in use provide for a number of more or less complicated features that fail to solve the problem in an efficient and economical way. None of these patents or prior art suggest the novel features of the present invention.

A brief abstract of the technical disclosure in the specification and title are provided as well for the purposes of complying with 37 CFR 1.72 and are not intended to be used for interpreting or limiting the scope of the claims.

Without limiting the scope of the invention, a brief summary of some of the claimed embodiments of the invention is set forth below. Additional details of the summarized embodiments of the invention and/or additional embodiments of the invention may be found in the detailed description of the invention below.

### H. BRIEF SUMMARY OF THE INVENTION

It is one of the main objectives of the present invention to provide a comprehensive health tracking system to assist in the evaluation, consolidation and management of health needs across life’s spectrum.

It is another objective of this invention to provide a health inventory for patients and healthcare providers to use together to promote continuity and integrate health information with the focus of optimizing well-being.

It is still another objective of the present invention to provide a comprehensive, interactive, dynamic, audiovisual system to identify health changes or improvements and provide that information to both patient and health care provider.

Another objective of the present invention is to keep a patient informed about their health condition and to better partner with their health care team.

It is yet another object of this invention to provide such a software and hardware system, and the resulting process of improving healthcare, that is inexpensive to deploy, use and maintain while retaining its effectiveness.

Further objects of the invention will be brought out in the following part of the specification, wherein detailed description is for the purpose of fully disclosing the invention without placing limitations thereon.

These and other embodiments which characterize the invention are pointed out with particularity in the claims annexed hereto and forming a part hereof. However, for a better understanding of the invention, its advantages and objectives obtained by its use, reference can be made to the drawings which form a further part hereof and the accompanying descriptive matter, in which there are illustrated and described various embodiments of the invention.

### I. BRIEF DESCRIPTION OF THE SEVERAL VIEWS OF THE DRAWING

With the above and other related objects in view, the invention exists in the details of construction and combination of parts as will be more fully understood from the following description, when read in conjunction with the accompanying drawings in which:

FIG. 1 shows a diagram demonstrating an example of information topography.

FIG. 2 shows an example of the system running on a healthcare provider’s portable electronic device or other computing device.

FIG. 3 shows an example of the system running on a patient’s portable electronic device.

### J. DETAILED DESCRIPTION OF THE INVENTION

While this invention may be embodied in many different forms, there are described in detail herein specific embodiments of the invention. This description is exemplary of the principles of the invention and is not intended to limit the invention to the particular embodiments illustrated and described.

For the purpose of this disclosure, like reference numerals in the figures shall refer to like features unless otherwise indicated or is obvious by context.

The subject device and software combination along with the resulting method of improving healthcare and tracking health is sometimes referred to as the device, the invention, the system, the method, the health tracker, the health inventory system, the machine or other similar terms. These terms may be used interchangeably as context requires and from use the intent becomes apparent. The masculine can sometimes refer to the feminine and neuter and vice versa. The plural may include the singular and singular the plural as appropriate for a fair and reasonable interpretation that the situation may allow.

In one aspect, the invention may be characterized as a secure information management, organization and delivery platform. It is a comprehensive health tracking system that assists both the patient and the physician or healthcare provider to evaluate and manage health needs across the patient’s life spectrum.

A software application is central to the inventive concept where a health inventory is available to the patient and a plurality of health care providers that promotes continuity and integrates health information with a focus of optimizing the well-being of the patient.

It is important for a patient to be able to communicate back and forth with several health care providers and also to ensure that the health care providers have access to relevant information about that patient to mitigate risks and enhance continuity of care for the patient.

To the extent necessary to provide superior care, the health care providers should have adequate and timely access to information relating to the patient generated by other health care providers. Equally, it is important that the patient have access to all of this information to allow them to better stay informed and help themselves while navigating the health care process.

Throughout an individual’s life they may interact with numerous types of health care providers. Each of these health care providers bears responsibility for their own discipline. A patient’s overall well-being can be improved when these several health care providers can coordinate analyses, observations, treatments and plans for improving the condition of that patient.

For example, an ear nose and throat otolaryngologist (ENT) may consider information generated by an allergist or rheumatologist when interacting with that patient. By having information from various medical specialties consolidated in one place, and that is portable with the patient, an improved coordinated course of care is achievable.

The graphical user interface of the system may be oriented to specific body parts and body systems to aid in an easier understanding, particularly for the patient. For example, a designator for the head may include reports from ophthalmology, optometry, ear nose and throat, dental, neurosurgery or oncology. Similarly, another section may include thoracic areas in the form of systems, such as pulmonology, cardiology and musculoskeletal. Further, by way of example, other areas may include more generalized categories such as nutrition, dermatology, neurology, psychiatry, endocrine, behavioral, mental health and vascular.

Medical records may be added to the health inventory with each new patient visit. In one aspect the medical records are in a medical locker that is safeguarded from any unauthorized access those that do access with authority may be logged to ensure that the access was appropriate.

Certain medical records that are more sensitive may require additional authentication prior to access. For example, records relating to sexually transmitted diseases or of mental health issues may not be available to all health care providers that access the system.

A patient’s health inventory records may be initially set up by the primary care physician’s office. Typically, this could include a family and social history to assess risks particular to that patient including past medical, surgical, family and societal histories. For example, this may include a family history of cancers or particular maladies experienced by others genetically related to the patient. There may also be specific records as to prior surgeries, procedures, testing, vaccines or other interactions with the health care system for that patient.

The records are maintained on the secure server so that any health care provider who accesses these records will be able to obtain the most current version. This has a significant advantage over each health care provider maintaining an independent chart that is not accessible by other treating providers.

Because the patient owns the medical records and information in their health inventory, the patient is in control of who has access to those records. If a patient changes from one treating physician to another, that patient can restrict access from doctors no longer treating and provide new and complete access to a newly treating physician.

A valuable characteristic of the system is that it is able to integrate with medical document and record management systems of many third-party document management systems. The health inventory system preferably is able to integrate different document formats and styles into a uniform format that is able to be provided to health care providers that did not originate the documents. It pushes the information and populates the respective HMS.

To integrate differing medical record collection systems the health inventory system may be able to translate incoming documents into a universal format. Equally, as records are delivered to the other document management systems, those documents can be formatted appropriately for access by those systems.

For example, if a primary care physician’s office uses a first brand of document and patient records management software and a regional hospital system uses a second round of document management software, the health inventory system will ensure that documents received and stored from one provider will be accessible and storable in the other provider’s system.

Another aspect of the system provides for efficient communication between the patient and their health care providers. There may be messaging modules that allow real-time communication between a physician’s office of a patient. These may take the form of a traditional email, SMS -type systems or video communication platforms for a face to face virtual visit.

An appointment scheduling system may also be included. A calendaring system for both the patient and physicians will help ensure that schedules are coordinated and appointments are kept. This may include a reminder system to ensure that patients timely show up for appointments and that physicians are available to timely see scheduled patients.

The communications module may also include a message chat, voice chat and video communication module the patient and health care provider will then be able to see each other face-to-face for virtual visits. This may also be valuable if the patient wants to show the health care provider a particular condition. For example, it may be helpful for a dermatologist to see a skin condition or for an occupational therapist to observe the healing of a wound. Audio, video and network communication hardware native to the user’s portable electronic device on which the system operates may be used.

An advantage of this health inventory system is that the basic and demographic information that is often required by physician’s offices on initial intake may be gleaned directly from the system. In this way, when a patient sees a new provider they do not need to fill out the same initial forms repeatedly. This initial information, such as contact information for the patient, insurance or payment information, allergies and other basic information may be gleaned from a standardized form suitable to all sub-specialties.

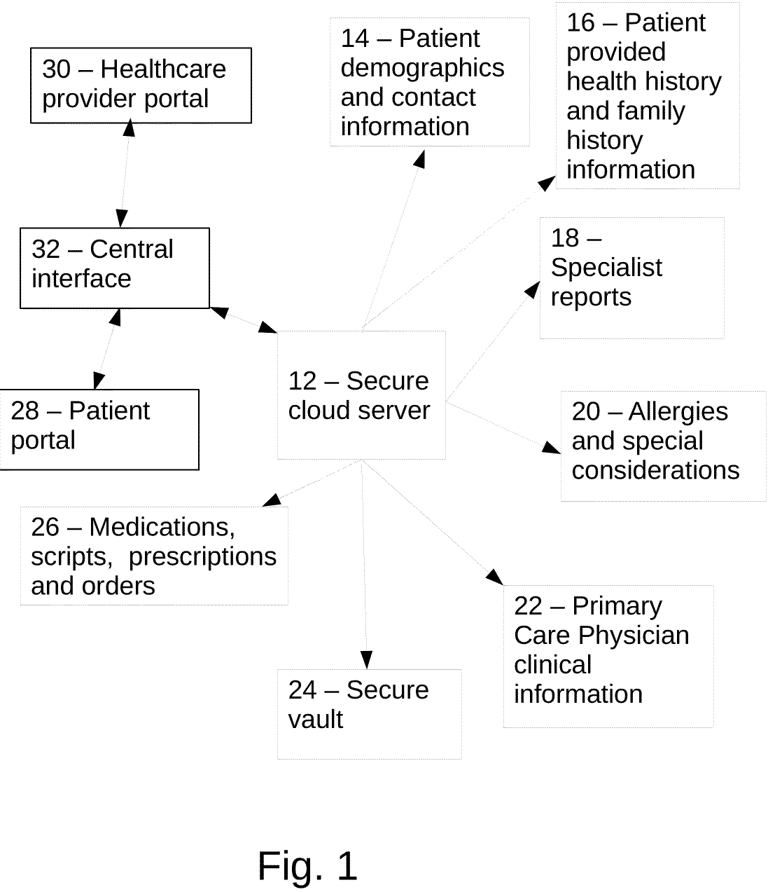

Looking now at FIG. 1, an example of a general computing topography is provided. The secure cloud server 12 stores and accesses information subgroups such as patient demographics and contact information 14, patient provided information family history 16, specialist reports 18, allergies and special considerations 20, primary care physician clinical information 22, a secure vault 24 and medications prescriptions, scripts and orders 26. Other categories and folders may be provided as needed and remain within the scope of the inventive concept.

Patient demographics and contact information 14 may include, information requested and required by most physician’s offices upon initial intake. This may include contact information, allergies and sensitivities, medications, surgical history and particular special needs of the patient’s that may be useful for any treating health care provider.

By having this common data set available to all health care providers the initial intake at any new physician’s office will be less burdensome on the patient. This also may include insurance, medicare and medicaid information that could be applied to any medical visit and is required by most doctor’s offices. This also facilitates the clinician’s review prior to interacting with the patient to allow better preparation.

This common data set may also include special alerts, such as hearing or vision impairment, limited language proficiency or whether the patient is guardian dependent. This can help to reduce healthcare delivery delays, improve efficiency and collaboration between patients and healthcare providers.

The group of information characterized as patient provided information family history 16 may also be stored on the secure cloud server 12. This may include documents that the patient wants to provide to the physician including information that may not be directly attributable to the patient. For example family history of certain conditions may be relevant to the treating physician. Although family history may be non-contributory to the patient’s acute healthcare visit or for certain specialists, it still plays a role in the health history intake.

Another subcategory of specialist reports 18 may be grouped together on the secure cloud server 12. Generally, specialist reports may include surgical reports, allergist reports, radiography and specialists’ consultant notes. The specialist reports 18 may also be further sub-categorized into data sets useful for a particular treatment.

For example, a fertility treatment may include specialty lab reports, psychology, ultrasound radiography and genetic assessments. Having these subgroups together can be useful for organizational purposes. It may also limit access to groups of information that may not be needed for a particular treatment. Continuing this example, the fertility treatments may not be particularly relevant to a dental assessment. Therefore, the dental office would have little need to access these files.

Another category of particular interest is allergies and special comsiderations 20. In this example allergies and special considerations 20 are separated out as of particular importance to any type of medical provider. By having special considerations and allergies separate and easily identifiable they are more readily referenced.

Allergies and special considerations 20 may contain specific things that the patient reports as not well-tolerated. Special considerations may include patient reported and medically determined things that may be of particular importance to the treating physician. For example, if certain medications or a course of treatment is preferred by the patient or otherwise not well-tolerated by the patient, these important notes may be placed in the allergies and special considerations 20 section.

Primary care physician clinical information 22 may be placed in its own folder. Generally, primary care physician notes are those generated most frequently by the patient’s primary treating doctor. These are going to be the most relevant for day-to-day care of the general health and well-being of the patient. These may include wellness visit notes and routine blood work. The primary care physician clinical information 22 folder is likely the first place that a primary care physician would look to review patient files prior to a routine clinical appointment.

Secure vault 24 may contain information that is more sensitive and may not be needed by general health care providers. For example, this may include mental health treatments and conditions and communicable disease information. There may be a separate or additional login for a health care provider reviewing this information to ensure that it is only provided on a need to know basis to ensure the privacy of the patient and track access.

Medications prescriptions and orders 26 may be a convenient location to store this type of information. This particular folder may be shared with pharmacists and other treating physicians to ensure that there are no contraindications, medical conflicts or duplicate prescriptions for the patient.

The secure cloud server 12 stores all the patient information in a digitally secured environment. This information is only accessible through the secure central interface 32. The information contained on the secure cloud server 12 may be encrypted and any access attempts to this information may be logged to ensure compliance with HIPAA or other medical documentation and security standards.

The central interface 32 is the backbone of the computing and software power of the system. The central interface 32 controls the aspects of security, login, access and distribution of any information held by the health inventory system. The central interface 32 is essentially the primary software platform operating the health inventory system. It may be run on a specific server or may be more generally characterized as a cloud computing server.

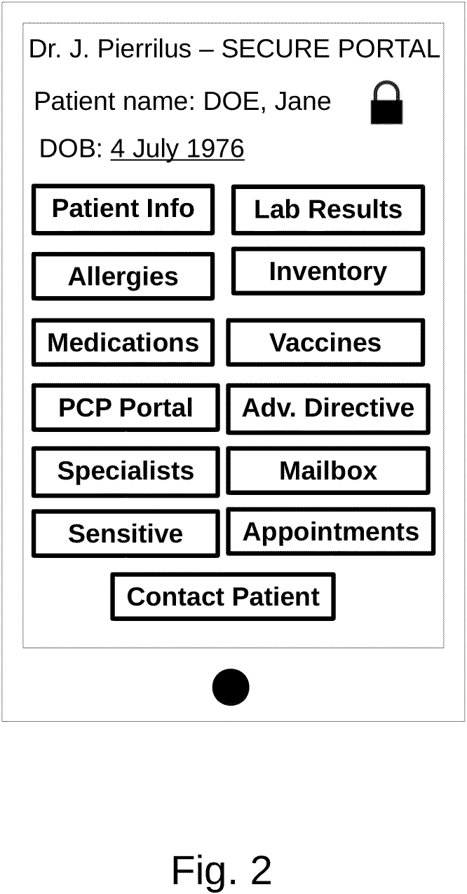

The health care provider portal 30 is the access point for physicians or other health care providers. An example of an interface screen for the health care provider portal 30 is shown in FIG. 2. The health care provider portal 30 interacts directly with the central interface 32 to deliver and retrieve documents and other information through the secure cloud server 12.

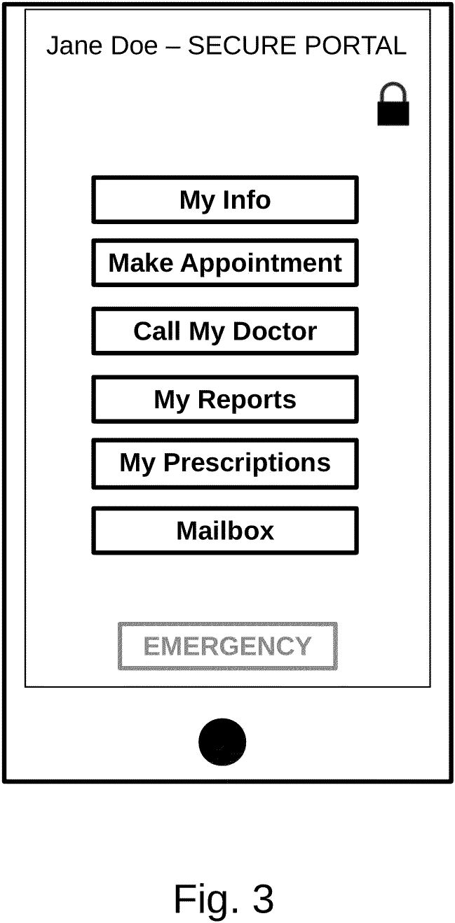

The patient portal 28 is the access point more suited for patient interaction. The patient portal directly connects to the central interface 32 that in turn sends and receives documents and other information through the secure cloud server 12. An example of a patient portal 28 is shown in FIG. 3 and described below.

Looking at FIG. 2, an example of a secured physician portal is shown. An icon of a padlock indicates that the session is secure and there is a secure communications link between the health care providers electronic device and the central interface 32. To help ensure accuracy, the patient’s name and their date of birth is identified on the main screen. The health care provider can selectively enter the information page for patients registered and associated with her practice.

The showing of options in FIG. 2 are merely exemplary of what the health care provider who owns this device may frequently need. This health care provider may optionally have settings to adjust the screen to quickly link to the information that she may need for a particular patient by showing more or fewer options.

Continuing this example, patient information can be directly accessed. The health care provider can review and amend information as needed. An allergies link is also provided that may provide sensitivities and special needs for this particular patient. This information is readily accessible to the physician.

Medications and vaccines can also be easily accessed from this primary screen. Sensitive information is contained in a separate area that requires specific and intentional entry into and may require an additional login or credential verification. Lab results, general inventory and specialist reports may also have direct links on the screen to obtain those documents and information.

Legal documents such as an advance directive, medical power of attorney and organ or tissue donation authorization may be contained in a separated folder.

Patient health care provider communication is an important aspect of the health inventory system. Ways to communicate between the physician and the patient is manifest by a mailbox where messages can be sent and received between the parties. Appointments can also be scheduled and amended in an appointment and calendar section. For ease of communication the health care provider may also directly choose to contact the patient. This may be through direct messaging, email, by direct telephone link or by providing instructions to a nurse or other staff member to contact the patient.

FIG. 3 shows an example of the secure portal as viewed by the patient. Generally, the patient portal is simplified for ease of access by the patient. The patient may adjust the look of the portal as desired to show more or fewer direct links to the documents and data contained in the health inventory system.

In this example shown in FIG. 3, the patient has the ability to amend their personal information. This may include revising insurance information, mailing addresses, billing information or other changing personal conditions.

The patient’s prescriptions and specialist orders may be held in a easy to locate folder with a direct link on the main screen. This can assist a patient with providing the correct and timely information to pharmacists, specialists or other health care providers.

Continuing this example for FIG. 3, and like the health care provider portal shown in FIG. 2, a key element of the health inventory system is to facilitate communication between patient and health care provider. There may be direct links to make an appointment with the health care provider or to make direct calls to the health care providers office.

A mailbox or other communication aspect can provide direct messaging between the interested parties. This mailbox may be linked to an external general email to provide a copy of the actual content or a notification to have the patient access the secure health inventory system for more secure communication.

In some situations it may be advantageous for the patient to be able to rapidly access emergency responders. In situations of acute distress, where the patient needs immediate help, it may be more appropriate to have emergency services come directly to the aid of the patient. Essentially this is calling “911”. By pressing the emergency button the patient can have immediate audio or audiovisual contact with the emergency services as well as provide location information derived from GPS from their portable device provided to the paramedics.

An important version of the invention can be fairly described as a process for coordinating a patient’s care information inventory between multiple healthcare providers comprised of the steps including where first the patient establishing a patient account on a server. The patient owns this account and can take the information with her wherever she is and can allow access to healthcare providers she selects. The patient accesses the patient account through a patient portal on her computing device and creates a body of first information and records the first information on the server through the patient portal. The first information is comprised of: a patient history, a family history, a demographic information and a contact information or any other information known by the patient. A first healthcare provider creates a second information and accesses the patient account on the server through a healthcare provider portal and records the second information to the patient account on the server. The second information directly relates to the patient and is comprised of at least three (or up to five) selected from: a specialist report, allergies, special considerations, primary care clinical information, medications, scripts, prescriptions and orders. A second healthcare provider accesses the first information and the second information in the patient account on the server through the healthcare provider portal. The second healthcare provider creates a third information and accesses the patient account on the server through the healthcare provider portal and recording the third information to the patient account on the server. The third information directly relates to the patient and is comprised of at least three (or up to five) selected from: a specialist report, allergies, special considerations, primary care clinical information, medications, scripts, prescriptions and orders. Preferably, communication between the patient, all healthcare providers, the server, the patient portal and the healthcare provider portal are digitally secure and encrypted to comply with privacy rules and regulations. Optionally, the patient selectively allows access to specific healthcare providers in the patient account as needed, for example to bring on another specialist, while traveling or when using a different physician.

The foregoing description conveys the best understanding of the objectives and advantages of the present invention. Different embodiments may be made of the inventive concept of this invention. It is to be understood that all matter disclosed herein is to be interpreted merely as illustrative, and not in a limiting sense.

I/we claim:

1. A process for coordinating a patient’s current care information inventory between multiple healthcare providers comprised of:
the patient establishing a patient account on a server;
wherein the patient account is owned by, and is portable with, the patient;
the patient accessing the patient account on the server through a patient portal and creating a first information and recording the first information on the server through the patient portal;
wherein the first information is comprised of: a patient history, a family history, a demographic information and a contact information;
a first healthcare provider creating a second information and accessing the patient account on the server through a healthcare provider portal and recording the second information to the patient account on the server;
wherein the first healthcare provider may access or record any information in the patient account only with authority from the patient,
wherein the second information directly relates to the patient and is comprised of at least three selected from: a specialist report, allergens, special considerations, primary care clinical information, medications, prescriptions and orders;
a second healthcare provider accessing a current version of the first information and the second information in the patient account on the server through the healthcare provider portal;
the second healthcare provider creating a third information and accessing the patient account on the server through the healthcare provider portal and recording the third information to the patient account on the server;
wherein the second healthcare provider may access or record any information in the patient account only with authority from the patient,
wherein the second information directly relates to the patient and is comprised of at least three selected from: a specialist report, allergens, special considerations, primary care clinical information, medications, prescriptions and orders.

2. The process for coordinating a patient’s care information inventory between multiple healthcare providers in claim 1 further characterized in that communication between the patient, all healthcare providers, the server, the patient portal and the healthcare provider portal are digitally secure.

3. The process for coordinating patient’s care information inventory between multiple healthcare providers in claim 1 further characterized in that the patient selectively allows access to any of the first second or third information to any of the first or second healthcare providers.
